(19) United States
(12) Plant Patent Application Publication
(10) Pub. No.: US 2012/0185981 P1
(43) Pub. Date: Jul. 19, 2012

(54) Poinsettia plant named 'NPCW11190'

(50) ;

(75) Inventors: Nils Klemm, Stuttgart (DE); Guido von Tubeuf, Stuttgart (DE)

(73) Assignee: KLEMM+SOHN GMBH & CO.KG, Stuttgart (DE)

(21) Appl. No.: 12/930,882

(22) Filed: Jan. 19, 2011

(51) Int. Cl. A01H 5/00

(52) U.S. Cl. PLT/307

(57) ABSTRACT

A new poinsettia plant named ‘NPCW11190’ particularly distinguished by its medium vigor, strong stems and upright growth habit, oak-leaved bracts with a lasting brilliant dark red color, and very early flowering date, is disclosed.

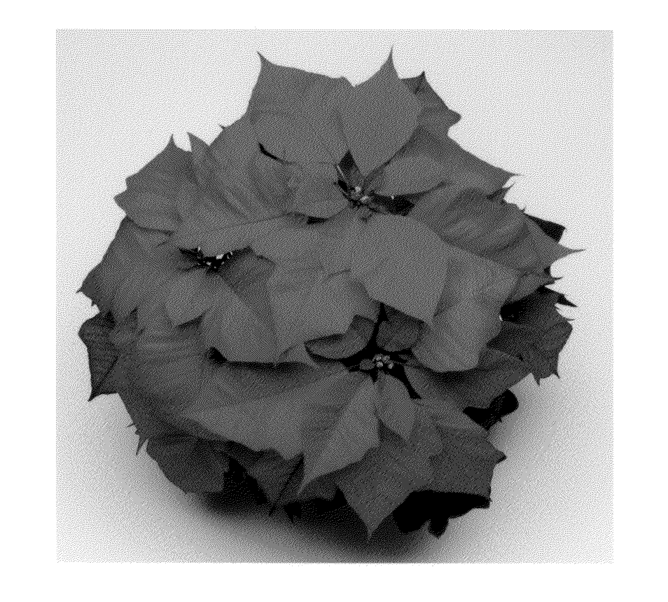

### GENUS AND SPECIES

Euphorbia pulcherrima Willd. ex Klotzsch

### VARIETY DENOMINATION

‘NPCW11190’

### BACKGROUND OF THE NEW PLANT

The present invention comprises a new and distinct variety of poinsettia plant, botanically known as Euphorbia pulcherrima Willd. ex Klotzsch and hereinafter referred to by the variety name ‘NPCW11190’. The new variety is the result of a cross conducted in December of 2005 in Kenya between the female parent poinsettia plant ‘X 017’ (unpatented) and the male parent poinsettia plant ‘X 212’ (unpatented). A single plant selection was subsequently chosen for further evaluation and asexual propagation.

‘NPCW11190’ was first propagated via vegetative cuttings in the spring of 2007 and has been asexually reproduced by vegetative cuttings in Stuttgart, Germany for over six generations. ‘NPCW11190’ has been found to retain its distinctive characteristics through successive asexual propagations via vegetative cuttings.

Plant Breeder's Rights for this variety were applied for in Canada on Dec. 15, 2010. ‘NPCW11190’ has not been sold or made publicly available more than one year prior to the filing date of this application.

### SUMMARY OF THE INVENTION

The following are the most outstanding and distinguishing characteristics of this new variety when grown under normal horticultural practices in a glass greenhouse in Stuttgart, Germany.

1. Medium vigor with strong stems and upright habit;

2. Oak-leaved bracts with a brilliant dark red color that doesn't fade; and

3. Very early flowering date.

### DESCRIPTION OF THE PHOTOGRAPH

This new poinsettia plant is illustrated by the accompanying photograph which shows the overall plant habit including blooms and foliage of the plant. The colors shown are as true as can be reasonably obtained by conventional photographic procedures. The photograph is of a whole plant about four months-old and in full flower.

### DESCRIPTION OF THE NEW VARIETY

The following detailed description sets forth the distinctive characteristics of ‘NPCW11190’. The data which define these characteristics were collected from asexual reproductions carried out in Stuttgart, Germany. The plant history was taken on four month-old plants grown in 13 centimeter pots from August to December with a minimum pinch date of 32 weeks. The plants were grown in a greenhouse covered with glass. Color readings were taken under natural light in the greenhouse. Color references are primarily to the R.H.S. Colour Chart of The Royal Horticultural Society of London (R.H.S.), Fifth Edition (2007).

### DETAILED BOTANICAL DESCRIPTION OF THE NEW PLANT

Classification:
   Family.—Euphorbiaceae.
   Botanical.—E. Pulcherrima Willd. ex Klotzsch.
   Common.—Poinsettia.
Parentage:
   Female parent.—‘X 017’ (unpatented).
   Male parent.—‘X 212’ (unpatented).
Growth:
   Time to produce a rooted cutting.—4 weeks.
   Blooming habit.—Intermittent; blooms from November to January.
   Flowering response time.—Approximately 55 days from beginning of short days until flowering.
   Keeping quality.—Very good.
Plant:
   Form.—Triangular.
   Growth habit.—Upright.
   Height.—22.0 cm.
   Width.—35.0 cm.
   Number of branches.—6.
   Average number of inflorescences per plant.—5 to 6.
Stems:
   Color.—Lower part: RHS 138A Upper part: RHS 144B.
   Length.—2.0 cm.
   Internode length.—0.5 cm to 2.0 cm.
   Diameter.—0.4 cm to 0.5 cm.
Leaves:
   Quantity.—6 to 10 per lateral branch.
   Arrangement.—Alternate.
   Shape.—Ovate.
   Apex.—Acute.
   Base.—Rounded.
   Margin.—Serrated.
   Lobes.—Medium.
   Texture (both surfaces).—Smooth.
   Vein color.—Upper surface: RHS 46A (leaf base) Lower surface: RHS N 34C.
   Variegation.—No.
   Size.—Length: 8.0 cm to 12.0 cm Width: 5.0 cm to 7.0 cm.
   Color.—Mature foliage: Upper surface: RHS 139A Lower surface: RHS N 138B Immature foliage: Upper surface: RHS 143A Lower surface: RHS 138B.
   Leaf petiole.—Length: Approximately 4.0 cm Diameter: Approximately 0.2 cm Color: Upper surface: RHS 46A Lower surface: RHS N 34A Texture: Smooth.
Inflorescence:
   Lastingness of inflorescence on the plant.—3 to 4 weeks.
   Diameter.—Approximately 20.0 cm.
   Height.—2.0 cm to 3.0 cm.
Bracts:
   Number of bracts per inflorescence.—5 to 6.
   Shape.—Ovate.
   Apex.—Acute.
   Base.—Rounded.
   Margin.—Medium lobes.
   Size.—Length: 9.0 cm to 11.0 cm Width: 5.0 cm to 6.0 cm.
   Texture (both surfaces).—Smooth.
   Vein color.—Upper surface: RHS 46A Lower surface: RHS 45C.
   Bract color.—Upper surface: Between RHS 45A and RHS 46A Lower surface: Between RHS 45B and RHS 45C.
   Bract petiole.—Length: 1.5 cm to 2.5 cm Diameter: 0.1 cm to 0.2 cm Color: Upper surface: RHS 46A Lower surface: RHS 45C.
Cyme:
   Diameter.—2.5 cm.
   Cyathia number.—4 to 5 per inflorescence.
   Cyathium.—Shape: Ovate Diameter: 0.3 cm to 0.5 cm Length: 0.4 cm to 0.7 cm Color: RHS 135C and RHS 142B.
   Peduncle.—Color: RHS 142B Length: 0.1 cm to 0.2 cm.
   Nectar cups.—Number: 1 per cyathia Diameter: 0.1 cm when closed, 0.2 cm when open Size: 0.3 cm to 0.5 cm Color: RHS 142C when closed, RHS 13B when open.
Reproductive organs:
   Stamens.—Quantity: 10 to 15 per cyathia Shape: Ovate Filaments: Length: 0.3 cm Color: RHS 39A Pollen: Quantity: Moderate Color: RHS 6A.
   Gynoecium.—Present.
Fruit and seed set: No fruit or seed have been observed
Disease and insect/pest resistance: Good

### COMPARISON WITH PARENTAL AND COMMERCIAL VARIETIES

‘NPCW11190’ differs from the female parental poinsettia plant ‘X 017’ by having medium sized foliage and an early season flowering date, while ‘X 017’ has small foliage and a mid-season flowering date.

‘NPCW11190’ differs from the male parent ‘X 212’ by having dark colored leaves and dark red bracts, while ‘X 212’ has green leaves and light red bracts.

‘NPCW11190’ differs from the commercial variety ‘Christmas Carol’ (unpatented) by having a bract color between RHS 45A and RHS 46A, while ‘Christmas Carol’ has a bract color between RHS 46B and RHS 46C. Additionally, ‘NPCW11190’ has thicker stems and bigger cyathias, while ‘Christmas Carol’ has thinner stems and smaller cyathias.

We claim:

1. A new and distinct variety of poinsettia plant named ‘NPCW11190’ as described and shown herein.
